(19) United States
(12) Patent Application Publication
(10) Pub. No.: US 2014/0012394 A1
(43) Pub. Date: Jan. 9, 2014

(54) HEMISPHERE FOR BLADDER EXPANSION IN PATIENTS WITH LOW COMPLIANCE

(71) Applicant: Antonio Sambusseti, Cremona (IT)

(72) Inventor: Antonio Sambusseti, Cremona (IT)

(21) Appl. No.: 14/002,200

(22) PCT Filed: Mar. 2, 2012
(86) PCT No.: PCT/EP12/53676

§ 371 Date: Sep. 23, 2013

(30) Foreign Application Priority Data
Mar. 11, 2011 (IT) MI2011A 000387

(51) Int. Cl. A61F 2/04

(52) U.S. Cl. CPC A61F 2/042
USPC 623/23.65

(57) ABSTRACT

A device for the expansion of an atrophied bladder formed by a hemisphere (100,200), as a single piece, in biocompatible material, characterized in that the material is selected from polylactic acid (PLA) and silicone coated with pyrolytic turbostratic carbon or with amorphous diamond-like carbon.

The present invention relates to a hemisphere for bladder expansion in patients with low compliance (low filling capacity) in the treatment and therapy of atrophied bladders.

Patients with low compliance generally have an atrophied bladder whose volume is about to 150-200 cc, i.e. much smaller than the volume of a healthy bladder which is normally around 400 cc. This entails, as is intuitive, serious problems for the patient.

In bladder expansion surgical procedures the replacement of the atrophied bladder with an artificial bladder, for example the one described in WO2009/077047, is a method generally not practised since the tissue of the atrophied bladder is not considered a damaged tissue.

In fact, in order to increase the available volume of an atrophied bladder use is normally made of a prosthesis with a hemisphere shape to be sutured on the incised atrophied bladder.

This hemisphere is made with the tissue of the intestine of the actual patient in order to have high compatibility and reduced rejection with reduced formation of the fibrous capsule.

However the tissue of the intestine does not always have the mechanical properties of the bladder such as elasticity, or the ability to assume stably a substantially hemisphere shape necessary for expansion of the bladder.

The patent application WO 2007/095193 describes an implant made up of a hemisphere covered by a population of cultivated, autologous or allogeneic cells, suitable for reconstructing, in a genetic laboratory, the three-dimensional structure of the tissue or of the organ which is then to be implanted in the patient. The hemisphere is therefore used as a support for depositing on its surface a population of cells cultivated in vitro.

This implant, which is only implanted after having been covered with cells, is somewhat complex, costly and lengthy to produce seeing that before the phase of covering with cells it is necessary to carry out a series of lengthy and complex preparatory phases: an initial phase of isolation of the cells to be cultivated by means of biopsy, a phase of growing of the isolated cell number, one of population as well as a phase of pre-treatment of the surface of the hemisphere so that it can be populated by the cells.

Moreover the aforesaid hemisphere has to provide fins, rings and handpieces necessary for a manipulation by the surgeon without damage to the tissue placed over it, thus making the construction of this hemisphere more complex.

Implants are also known with a planar or slightly curved shape suitable for the replacement of portions of bladder wall such as for example the patch described in WO 2007/039160 and the scaffold described in WO 2011/018300, which however, given their shape, cannot be used for bladder expansion in that they cannot be transformed into devices endowed with volume.

The object of the present invention is to eliminate, at least in part, the disadvantages of the prior art, by providing a device (implant) for bladder expansion in patients with low compliance, which is elastic but also with such rigidity as to be able to maintain the rounded shape of the bladder, once implanted, which is reliable without showing possible leaks of liquid and which is resistant to urine.

Another object of the present invention is to provide such a device which is also with zero rejection, with lack of adherence of the fibrous capsule and provided with high compatibility, which allows tissue reconstruction of similar quality to the original tissue.

Another object of the present invention is that of providing such a device which is simple and easy to manufacture and can be implanted in the patient without excessive preparatory phases.

These objects are achieved by a biocompatible hemisphere device according to the invention having the features listed in the annexed independent claim 1. Advantageous embodiments of the invention are disclosed by the dependent claims.

The device according to the invention for expansion of the atrophied bladder is made up of a domed hemisphere, elastic and flexible and internally hollow, having a predetermined volume, made as a single piece of a biocompatible material which guarantees the absence of fibrous capsule around it once implanted.

The biocompatible material is selected from PLA and silicone coated with pyrolytic turbostratic carbon or with amorphous diamond-like carbon. This hemisphere has smooth internal and external surfaces, even when coated with pyrolytic turbostratic carbon or amorphous diamond-like carbon. Moreover this hemisphere is lacking any covering by cultivated tissue cells and any surface treatment for encouraging the grafting of the growing tissues. In practice the aforesaid hemisphere is suitable for acting as scaffold only after insertion inside the patient, and for causing to grow on it only autologous cells from fibrous capsule, generated by the process of tissue reconstruction of the patient, which only takes place after its insertion.

Said hemisphere may have a plurality of holes, equally distanced and positioned on its perimeter rim projecting outwards (by way of a flange) in order to pass the suture thread through which is to fix said hemisphere to the non-removed bladder part.

The hemisphere shape of this device is imparted during the process of production of the same by means of moulding, during production and not during the operation phase.

Further features of the invention will be made clearer by the following detailed description, referred to one of its embodiments purely by way of a non-limiting example illustrated in the accompanying drawings, in which:

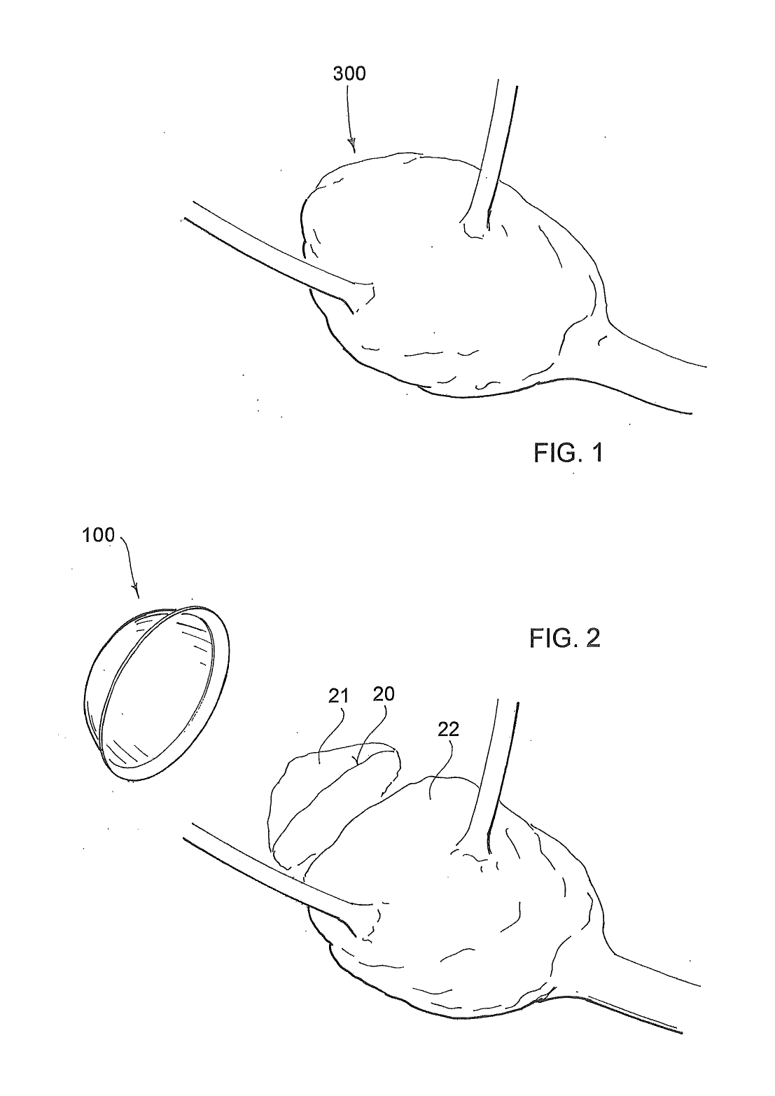

FIG. 1 is a perspective view of a bladder with low compliance with relative ureters and urethra;

FIG. 2 is a perspective view of the bladder of FIG. 1 wherein the upper part has been cut in order to be replaced by a hemisphere of the invention;

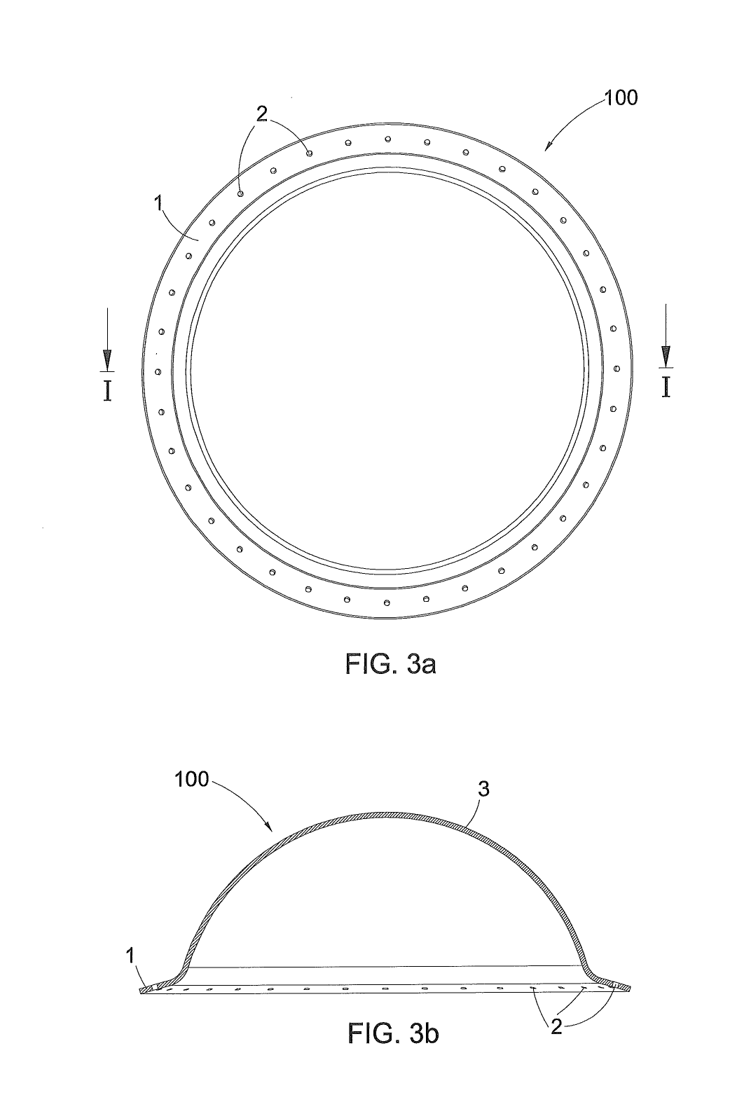

FIG. 3a) is a plan view from below of the hemisphere according to a first embodiment of the invention;

FIG. 3b) is a sectioned view of the hemisphere of FIG. 1a) taken along line I-I;

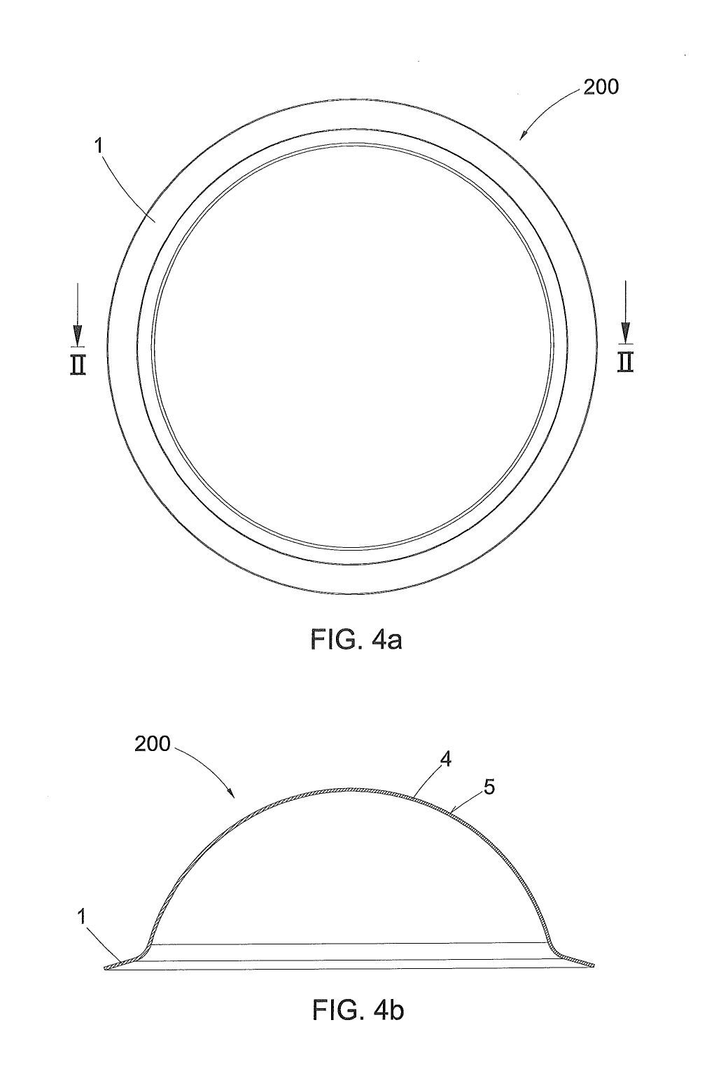

FIG. 4a) is a view from below of the hemisphere according to a second embodiment of the invention;

FIG. 4b) is a sectioned view of the hemisphere of FIG. 2a) taken along line II-II;

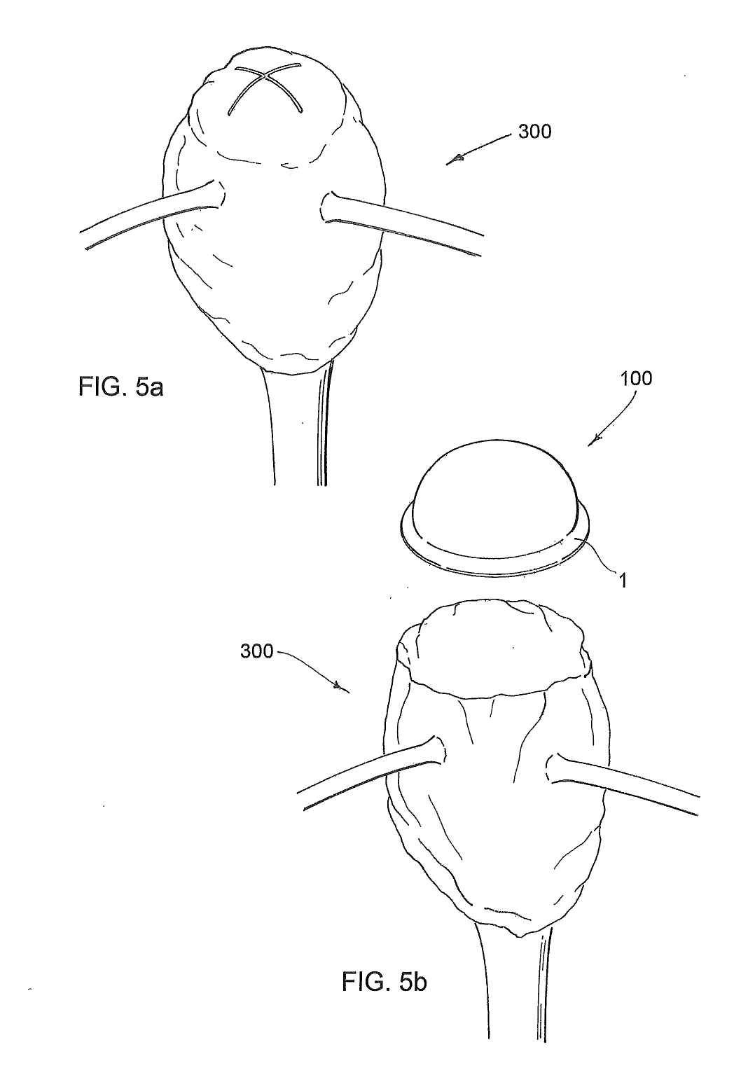

FIGS. 5a)-b) are perspective views of the atrophied bladder in the phases of expansion by insertion of the hemisphere.

Referring to FIGS. 3-4(a, b) indicated above, a hemisphere according to the invention is described, denoted overall by reference numeral 100.

The hemisphere 100 is internally hollow, has a circular profile in a plan view with diameter of about 80 mm and has a rim 1 projecting outwards and turned upwards.

In this first embodiment said hemisphere 100 is made of a biocompatible polymer which is also absorbable, constituted by the homopolymer or copolymers with base of lactic acid (L-, D-, racemic mixture, or dimer, esters, etc. or combinations thereof).

Particularly preferred is poly(D-lactide) acid or the poly(L-lactide-co-D,L-lactide) copolymer polyester (PLDLA, or otherwise identified also as PLDL or PLLA/PDLLA). This polyester is a copolymer having an L-lactide:D,L-lactide monomer composition of about 70:30. It is also possible to use a PLDLA copolymer as defined above, having a different monomer composition, for example with a monomer content of the L-lactide comonomer comprised between 70% and 30% (the D,L-lactide comonomer is the complementary part to 100).

Another example of polymer which can be used is poly-L-D-lactide acid, preferably having an L-lactide:D-lactide monomer composition of 70/30 or 50/50.

The aforesaid polymers with lactic acid base were found to be neutral when in contact with non-cultivated cells: this entails a rapid population of the device implanted by the cells of the growing surrounding tissue. At the same time the adhesion was found to be reduced due to the reduced interaction between these polymers and the biological molecules.

The thickness 3 of said hemisphere 100 is not binding for the purpose of the present invention: it is fairly reduced but is such as to ensure a sufficient rigidity such as to result in a self-supporting hemisphere, ensuring at the same time the elasticity and the flexibility necessary for the movements (dilations) of expansion and collapsing of the bladder due to the filling and emptying of the same.

Said thickness 3 may vary between 0.1 mm and 2 cm. In a preferred embodiment said thickness 3 is around 0.5-0.6 mm when the hemisphere is in silicone, while it is about 1 mm when it is made in PLA.

On the rim 1 of said hemisphere 100 in polylactic acid there is a plurality of holes 2 whose pitch is not binding for the purpose of the present invention and depends on the diameter of the holes 2. The diameter of the holes 2 may vary from a minimum to a maximum comprised between 0.1 and 3.0 mm.

In a preferred embodiment the holes 2 have a diameter of about 1 mm and are distanced with a pitch of 2.5 mm.

Said hemisphere 100 in PLA is obtained by means of moulding, even if it is possible to obtain it with other known techniques normally used for the formation of concave and hollow objects, formed in a single piece in a polymeric material.

In FIGS. 4a, 4b a second embodiment of the hemisphere in accordance with the invention is illustrated, denoted by the reference numeral 200.

Said hemisphere 200 has substantially the same diameter (or radius of the sphere) of the hemisphere 100, the same rim 1 turned upwards, yet it is made in silicone coated internally and externally with pyrolytic turbostratic carbon or amorphous diamond-like carbon (DLC).

Amorphous diamond-like carbon is a carbon coating, white or transparent, with stratified structure similar to the diamond (defined in fact as “diamond-like carbon”) with outstanding features of surface resistance such as hardness and resistance to abrasions, as well as being well tolerated by the skin and resistant to corrosion yet elastic.

Moreover it is neutral when in contact with cells and micro-organisms: this entails a rapid population of the device implanted by the cells of the growing surrounding tissue. At the same time the adhesion is reduced due to the reduced interaction between the coated surface and the biological molecules.

This coating of amorphous diamond-like carbon can also be “doped” with various compounds to achieve oil repellency or water repellency.

The coating in pyrolytic turbostratic carbon also has features of surface resistance, resistance to abrasions, resistance to corrosion. Moreover said pyrolytic turbostratic carbon was also found to be neutral when in contact with cells, resulting in a rapid population of the device implanted by the cells of the growing surrounding tissue.

At the same time the adhesion of the pyrolytic turbostratic carbon to the tissues is almost totally absent due to the reduced interaction between the coated surface and the biological molecules. In this way a substantial absence is obtained of the phenomenon of fusion to the surrounding tissues which takes place instead when other materials are used, for example a membrane of only silicone.

The silicone in fact has the tendency to co-penetrate with the polyprotein fibrous growth (red blood cells) and to fuse with the neotissues.

The tissues which are reconstructed around the present device, whether in PLA or in coated silicone, are moreover of similar quality to the original tissue, in particular they show substantially the same original elasticity.

The silicone used can be made up, for example, of copolymers of dimethyl and metavinyl siloxane, reinforced with silicon. A silicone for medical use is preferably used, such as for example that known by the code MED 4735™ and marketed by the company Nusil Technology.

Preferably said hemisphere 200 in coated silicone does not have holes 2 along the rim 1 since they can be made at the time of the suture of the hemisphere 200 being very elastic.

In practice the layer of silicone which constitutes the hemisphere 200 is formed by a membrane provided with sufficient flexibility, so as to ensure the proper functioning of the bladder.

The thickness 4 of said hemisphere 200 in silicone is preferably about 600 microns.

The thickness of the layer of coating 5 in pyrolytic turbostratic carbon or in amorphous diamond-like carbon is not binding for the purpose of the present invention and can be for example a microfilm of approximately 0.2-0.3 micron.

The application of said layer 5 of pyrolytic turbostratic carbon or of amorphous diamond-like carbon (DLC) is performed according to a known technique, such as for example PVD in the case of DLC.

The hemisphere 100,200 is prepared in a controlled environment that is to say with controlled contamination, in a white room. Once processing has finished, the hemisphere 100,200 is enclosed by a sheet of Tyvek to avoid contaminations, and sent to a cycle of sterilisation with base of ETO (ethylene oxide) or sent to a cycle of sterilisation with gamma rays (in the case of PLA). At this point the hemisphere is ready to be used in an operation.

The hemisphere 100, 200, without any covering with cultured cells, is applied to the bladder in place of the part removed according to known surgical techniques, after having sectioned the bladder and removed the upper portion, leaving the connections of the urethra and ureters to this bladder intact.

In fact the atrophied bladder 300 (FIG. 1) can be first cut into two parts, the upper part 21 whereof (FIG. 2) is removed while around the perimeter of the lower part 22 not removed the hemisphere 100 or 200 is sutured.

Alternatively, as illustrated in FIGS. 5a) and 5b), the atrophied bladder 300, comprising ureters and urethra, is only incised with a cross cut, opened and subsequently sutured to the hemisphere 100, 200 around the rim of the opening created by the cut. Over this hemisphere the neotissue will then form that comes from the natural growth of the polyprotein capsule around this implanted hemisphere, and therefore not coming from cultured cells.

For each of the embodiments of the hemisphere 100, 200 described above, suture threads to in absorbable or non-absorbable material can be used for the through suture.

In the case of the hemisphere in silicone coated with pyrolytic turbostratic carbon or amorphous diamond-like carbon, it is preferable to use absorbable thread in order to be able to remove easily the hemisphere 200 after a certain period, in general after 30 days, via simple withdrawal of the same from a side by means of removal in laparoscopy, or by removal in endoscopy, performed in day hospital or day surgery or day hospital with an operation, also without anaesthetic, lasting a few minutes.

In fact after the 30 days the hemisphere in coated silicone has to be removed in that at the end of the process of reconstruction of the bladder it falls inside it after the fixing suture, made with absorbable thread, has been absorbed.

The suture thread for the hemisphere 100 in PLA is also preferably in absorbable material, for example like the polymers mentioned above for the hemisphere 100 of the present invention, preferably PLA, PLLA. The reasons for this choice lie in the need for the hemisphere and sutures to be absorbed in the same timespan. The suture thread is then inserted in a round ¾ curved cylindrical needle, including the “Bassini” ones.

Other suture threads in bio-absorbable polymers in any case exist which could be conveniently adapted to the case in question and to the needs at the discretion of the surgeon.

However in the case of the hemisphere 100 in PLA the choice of the material of the suture thread is less important given the absorbability of the PLA after the 30 days.

The holes 2 for passage of the suture stitches do not constitute a risk of leaks of liquid, in that the tissue is reconstructed in a few hours. To avoid leaks of urine (liquid), the holes of the suture stitches can be sealed and closed with one cc (a drop) of surgical glue, such as for example Glubran 2™, normally available commercially.

One of the advantages of the hemisphere 100, 200 of the present invention is that it does not show any risk of adherence of the fibrous capsule both thanks to the coating in pyrolytic turbostratic carbon or amorphous diamond-like carbon which do not show any adherence with the growing tissues and to the absorbability of the PLA.

Moreover the hemispheres 100, 200 are resistant to urine and, in the case of the silicone, also considerably elastic.

Another advantage is represented by the fact that the present hemisphere can be used as an implant in the patient even without prior covering of its surface with cells cultivated in vitro, contrarily to what is taught by WO2007/095193, in light of the fact that the present hemisphere is able to act as a scaffold only after having been implanted inside the patient, causing to grow thereon only autologous cells from fibrous capsule, generated by the process of tissue reaction and reconstruction of the organism.

This entails a consequent saving in time and costs due to the non-use of machinery, apparatuses and staff highly qualified in genetics, tissue engineering and biology seeing that the cultured cells can come either from the patient or from a donor and be xenogeneic cells, or mixed, and that therefore these cells have to be obligatorily treated with immunodepressive therapy to make them compatible with the receiver.

It also has to be noted that in the art, for example in WO2007/095193, the neotissue or neobladder is made in a laboratory, under a hood, using the cultures positioned on the one or on the two hemispheres, including urethra and ureters, and that this neotissue or neobladder is implanted in the patient, removing the bladder completely, suturing urethra and ureters, and therefore the neobladder, or part thereof, does not have to form inside the patient in that it is already made in the laboratory, with cultured cells. In practice, seeing that the neobladder is already made when it is implanted, there is not tissue growth over these scaffolds of the prior art but only their absorption and the integration of the neobladder inside the patient.

Numerous detail modifications and changes, within the reach of a person skilled in the art, may be made to the present embodiments of the invention, in any case coming within the scope of the invention disclosed by the annexed claims.

1-8. (canceled)

9. An implant for increasing the volume of an atrophied bladder, said implant being constituted by a domed hemisphere device, internally hollow with a predetermined volume, made in a single piece in biocompatible material, the internal and external surfaces of said hemisphere are without covering from semination of cultured cells, characterised in that said material is silicone coated with pyrolytic turbostratic carbon or with amorphous diamond-like carbon, and in that said hemisphere is suitable for acting as a scaffold and for growing thereon autologous cells from fibrous capsule, generated by the process of tissue reconstruction of the patient, after its insertion inside the patient.

10. The implant according to claim 9 wherein the diameter of the hemisphere is about 80 mm.

11. The implant according to claim 9 wherein the hemisphere has a rim turned upwards.

12. The implant according to claim 9 wherein the hemisphere has a thickness ranging from 0.1 mm to 2 cm.

13. The implant according to claim 12 wherein the hemisphere has on the rim a plurality of holes, with a diameter of the holes comprised between 0.1 and 3.0 mm.

14. The implant according to claim 9 wherein the coating of pyrolytic turbostratic carbon or amorphous diamond-like carbon is a microfilm of about 0.2-0.3 micron.

15. The implant according to claim 9 wherein the suture thereof to the bladder is performed by using suture threads made of absorbable or non-absorbable material.

16. The implant according to claim 10 wherein the hemisphere has a rim turned upwards.

17. The implant according to claim 9 wherein the hemisphere has a thickness ranging from about 0.5-0.6 mm.
